(19) United States
(12) Patent Application Publication
(10) Pub. No.: US 2019/0010688 A1
(43) Pub. Date: Jan. 10, 2019

(54) Disposable Air Freshener

(71) Applicant: Terence Baxter, Syracuse, NY (US)

(72) Inventor: Terence Baxter, Syracuse, NY (US)

(21) Appl. No.: 15/644,629

(22) Filed: Jul. 7, 2017

(51) Int. Cl. E03D 9/00; A61L 9/012

(52) U.S. Cl. CPC E03D 9/005; A61L 9/012

(57) ABSTRACT

The current invention is a dissolving air freshening tablet for use in toilets. The air freshener is made of a rapidly dissolving material. The air freshener is placed in the toilet bowl before the toilet is used. As it dissolves it releases a film barrier on top of the water and a fresh smelling scent into the air. The user will choose when to use an air freshener, providing targeted application. A wide variety of scents can be produced, as well as basic chlorine tablets.

### CROSS-REFERENCES TO RELATED APPLICATIONS (IF ANY)

None.

### BACKGROUND

### 1. Field of the Invention

This invention relates to air fresheners and particularly ones for use in a toilet bowl.

### 2. Description of Prior Art

No one wants a house to smell like fecal matter, especially when guests are present or expected. To combat this, there are several types of air fresheners to mask or reduce bathroom odors. There are liquids that are dispensed from the bank tank, sprays, devices that deliver a consistent scent for a room, and scented cleaning agents.

There is still room for improvement in the art.

### SUMMARY OF THE INVENTION

The current invention is a strongly scented dissolving scented puck or tissue paper. It would be placed in the toilet before use, and releases a fresh scent as it dissolves.

The air freshener would be made of a material that rapidly dissolves in water, while releasing a scent. As the air freshener dissolves, it will also create a film barrier on top of the water.

Various scents can be produced. An unscented chlorine tablet option can also be made.

The product would be placed in the toilet bowl before the toilet is used.

### BRIEF DESCRIPTION OF THE DRAWINGS

Without restricting the full scope of this invention, the preferred form of this invention is illustrated in the following drawings:

FIG. 1 shows a line drawing with an air freshener puck in the foreground and a dispenser behind it;

FIG. 2 shows another view of the pucks and dispenser;

FIG. 3 shows a view of the pucks and dispenser;

FIG. 4 shows the dispenser from behind;

FIG. 5 shows the dispenser from above;

FIG. 6 shows the line drawing from below; and

FIG. 7 shows invention being used.

### DESCRIPTION OF THE PREFERRED EMBODIMENT

There are a number of significant design features and improvements incorporated within the invention.

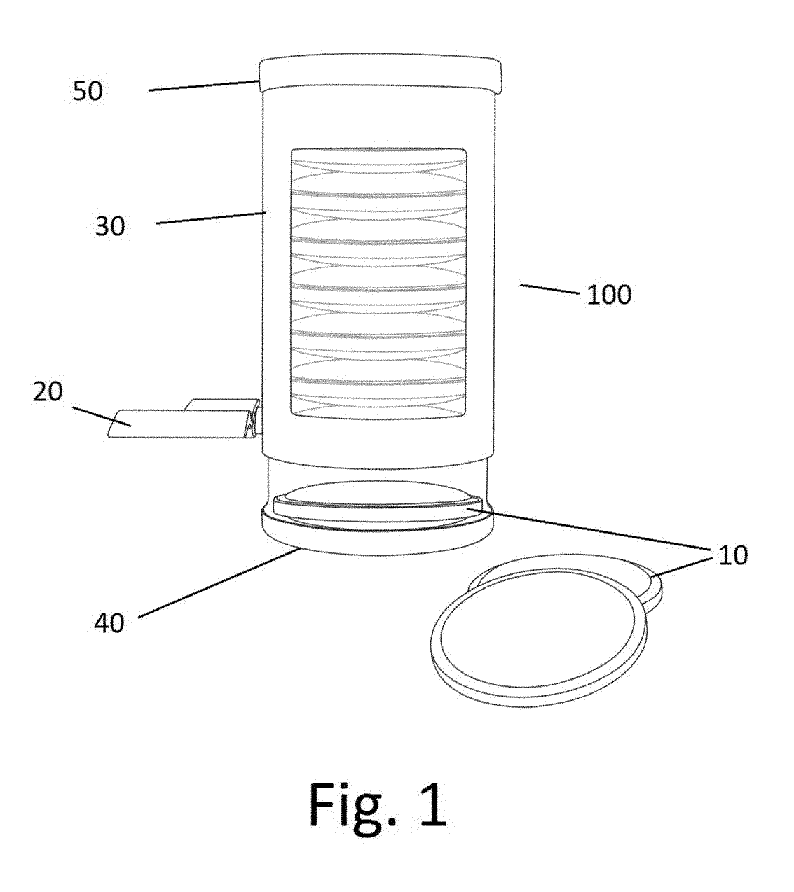

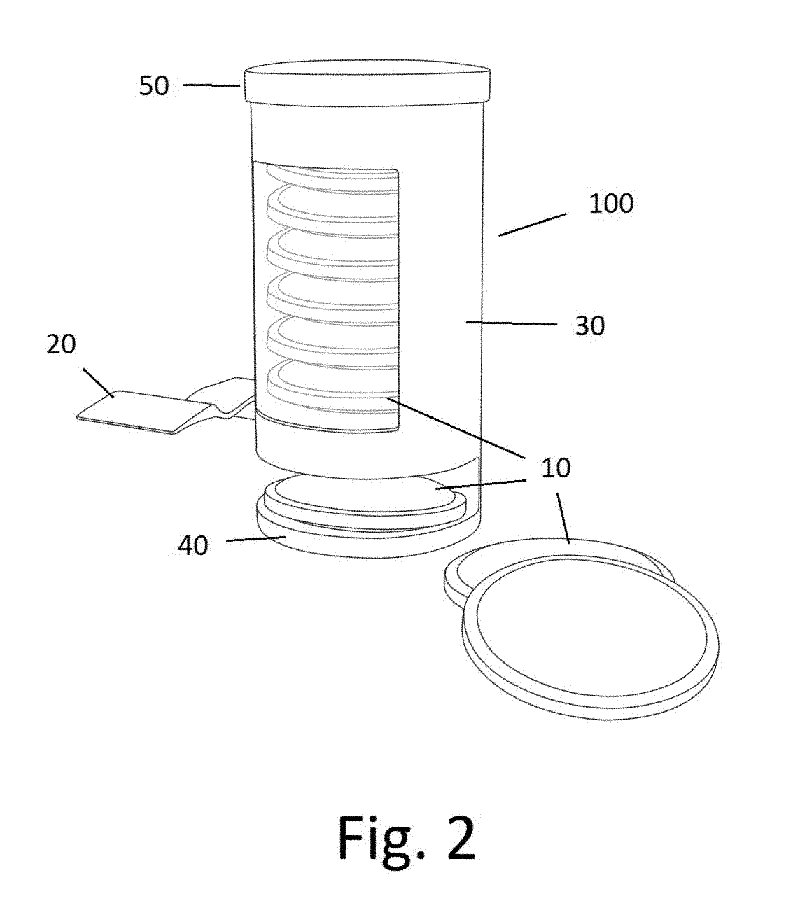

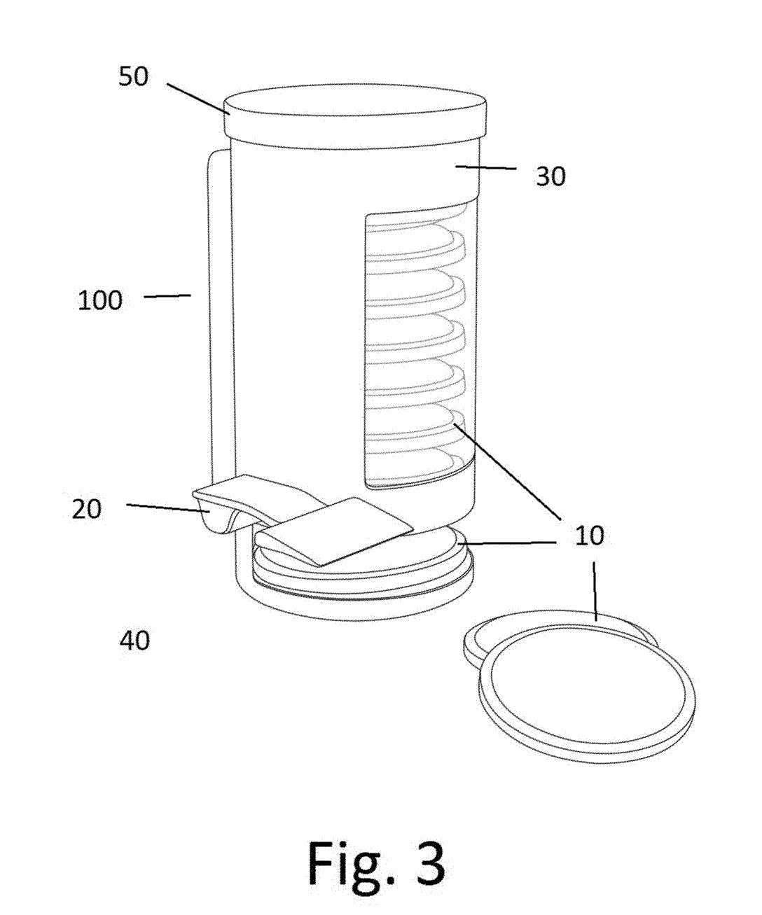

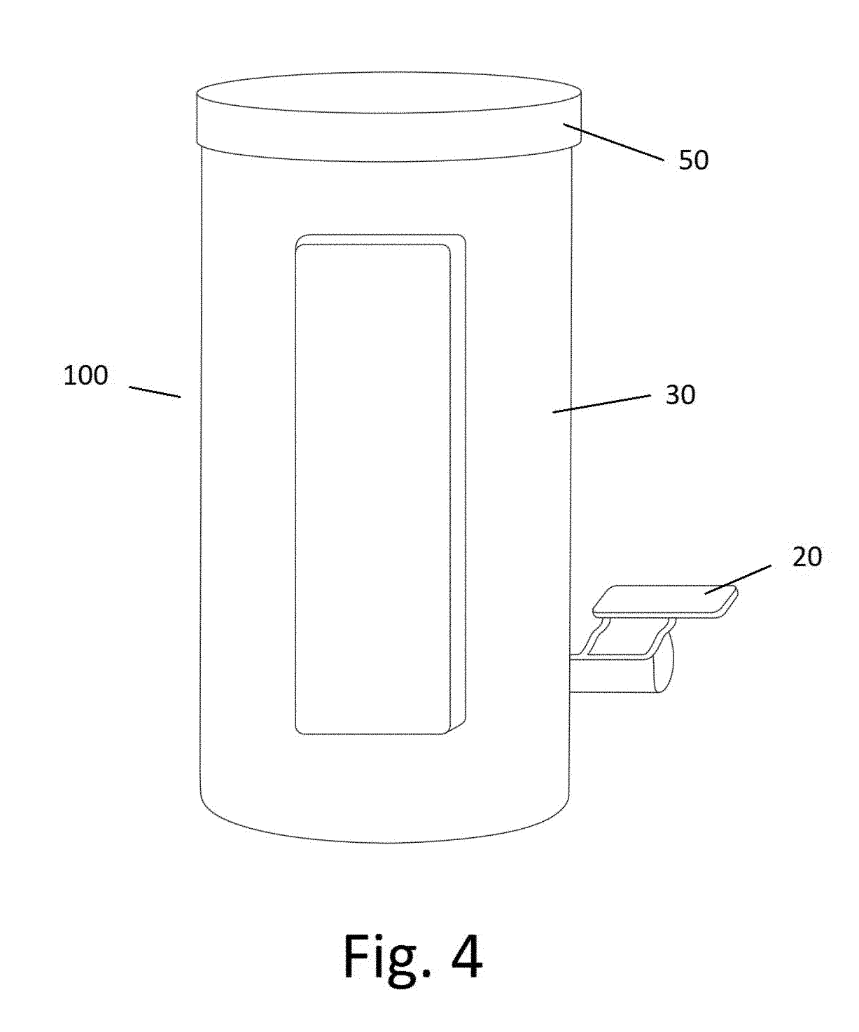

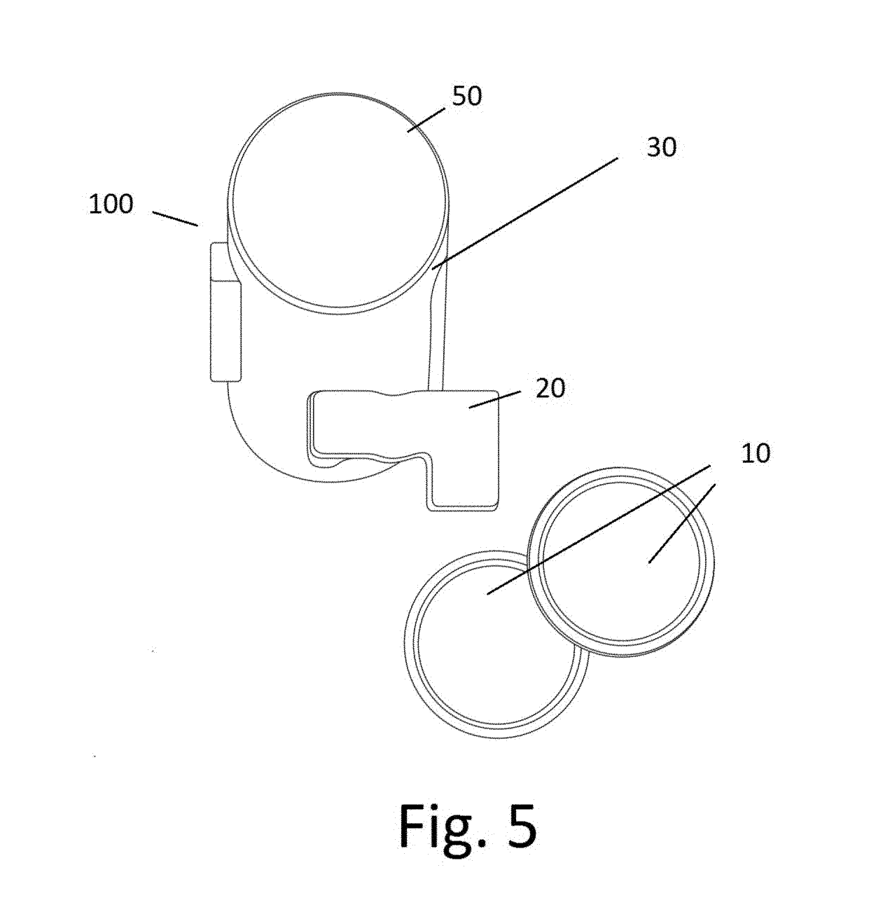

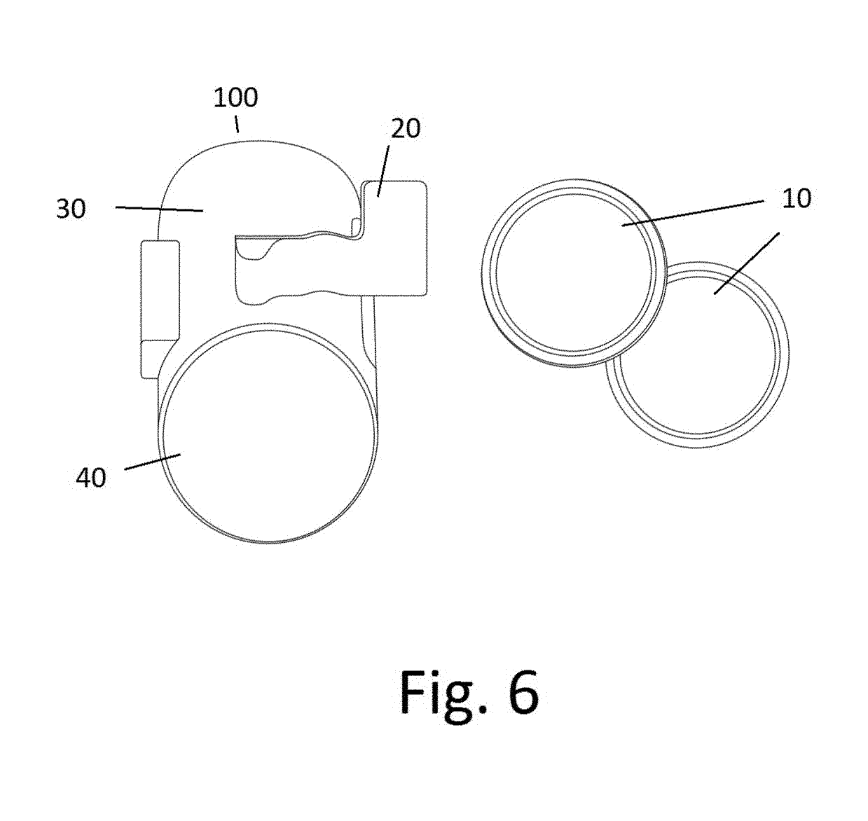

The current invention, as shown in FIGS. 1 through 6, is a strongly scented dissolving scented puck 10 or tissue paper 15. It would be placed in the toilet 200 before use, and releases a fresh scent as it dissolves.

The puck 10 or toilet paper 15 air freshener would be made of a material that rapidly dissolves in water, while releasing a scent. As the puck 10 or toilet paper 15 dissolves, it will also create a film barrier on top of the water.

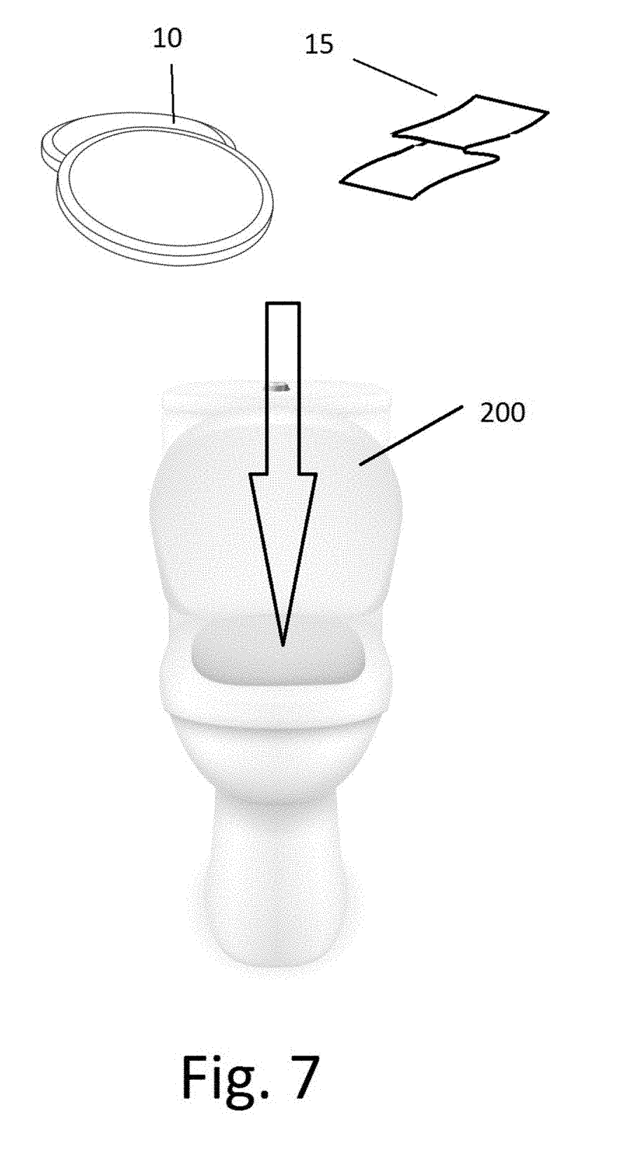

The puck 10 can be made with various scents or as an unscented chlorine tablet. The puck 10 or toilet paper 15 would be placed in the toilet bowl before the toilet is used as shown in FIG. 7. The puck 10 is a rapid dissolvable tablet infused and a scent or scents and dry chlorine. Once dropped into the water in the toilet bowl that puck 10 gives a burst of scent which serves to overpowers the smell of fecal matter.

The pucks 10 are designed in a shape that is easy to use and store. The rounded puck 10 are designed to be stored and dispensed from a dispenser 100 as shown in FIGS. 1 through 6.

The dispenser 100 has a base 40 on which a holding cylinder 30 is attached. The base 40 has an opening which gives the user access to the pucks 10. The pucks 10 are stacked in the cylinder 30 with the cylinder 30 having a slightly larger circumference then the pucks 10. The cylinder 30 is opened on the top which is covered by a lid 50. The pucks 10 are placed into the cylinder 30 through the opening at the top and stack against each other.

The dispenser 100 may have a dispensing handle 20 which when pressed releases a puck 10 to the opening in the base 40. In another embodiment, the puck 10 are stacked with the bottom puck 10 being level with the opening in the base 10 so a user just pulls the bottom puck 10 out.

### ADVANTAGES

The innovative air freshener provides a proactive, precision-based approach to bathroom air freshening. Unlike air freshening sprays, these tabs are used before smells are able to take hold. Unlike constant-release air fresheners and in-tank chemical dispensers, these products are used only when needed. It will address foul odors before they become a problem, without resorting to a constant release of chemical.

Although the present invention has been described in considerable detail with reference to certain preferred versions thereof, the point and scope of the appended claims should not be limited to the description of the preferred versions contained herein.

As to a further discussion of the manner of usage and operation of the present invention, the same should be apparent from the above description. Accordingly, no further discussion relating to the manner of usage and operation will be provided. With respect to the above description, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

That which is claimed is:

1. A device comprising:
a rounded puck that is dissolvable which has a scent and chlorine.

2. The device according to claim 1 further comprising:
Where the chlorine is dry chlorine.

3. The device according to claim 1 further comprising:
Where the puck is placed into a toilet bowl before using the bowl.

4. The device according to claim 1 further comprising:
Where the puck dissolves in the toilet.

5. The device according to claim 1 further comprising:
Where the puck is stored in a dispenser.

6. The device according to claim 5 further comprising:
Where the dispenser stores more than one puck.

7. The device according to claim 5 further comprising:
Where the dispenser has an opening and lid on the top where the puck is placed in the opening.

8. The device according to claim 5 further comprising:
Where the dispenser has a base and has an opening where the pucks are removed from the dispenser.

9. The device according to claim 5 further comprising:

10. A device comprising:
a toilet paper that is dissolvable which has a scent and chlorine.

11. The device according to claim 10 further comprising:
Where the chlorine is dry chlorine.

12. The device according to claim 10 further comprising:
Where the toilet paper is placed into a toilet bowl before using the bowl.
